(19) United States
(12) Patent Application Publication
(10) Pub. No.: US 2026/0114871 A1
(43) Pub. Date: Apr. 30, 2026

(54) ROBOTICALLY-ASSISTED DELIVERY AND POSITIONING OF MAGNETIC IMPLANTS FOR FORMING AN ANASTOMOSIS

(71) Applicant: GT METABOLIC SOLUTIONS, INC., Wilmington, DE (US)

(72) Inventors: Michel GAGNER, Montréal (CA); Todd A. KRINKE, Buffalo, MN (US); Thierry THAURE, San Jose, CA (US)

(21) Appl. No.: 19/155,399

(22) PCT Filed: Apr. 2, 2025
(86) PCT No.: PCT/US2025/022778

§ 371 Date: Aug. 11, 2025

Related U.S. Application Data
(60) Provisional application No. 63/574,713, filed on Apr. 4, 2024.

(51) Int. Cl. A61B 17/11; A61B 1/00; A61B 1/018; A61B 17/00; A61B 34/00; A61B 34/20; A61B 34/30; A61B 34/32

(52) U.S. Cl. CPC A61B 17/1114; A61B 1/00087; A61B 1/018; A61B 34/20; A61B 34/32; A61B 34/73; A61B 2017/00876; A61B 2034/2046; A61B 2034/301

(57) ABSTRACT

Robotic systems and methods for assisting in the delivering, navigating, guiding and/or positioning of magnetic implants configured for forming an anastomosis between two adjacent walls of a digestive tract are provided. The robotic system can include at least one a robotic arm engageable with a positioning tool. The positioning tool can include for instance an endoscope, a catheter, or a positioning wand. The positioning tool can contribute to positioning each of the magnetic implants at a corresponding target site of a desired anastomosis, such that the magnetic implants can magnetically couple to each other through the two adjacent walls of the digestive tract to form the anastomosis. Alternatively, the robotic system can include magnetic field generation device and associated external magnetic control assemblies to control a position of the magnetic implants.

### TECHNICAL FIELD

The technical field generally relates to medical techniques for treating digestive tract conditions. In particular, the technical field relates to robotically-assisted techniques for delivering and positioning magnetic implants for forming an anastomosis in the digestive tract.

### BACKGROUND

Metabolic surgeries and medical procedures to treat conditions associated with the digestive tract, diabetes and obesity often require alteration of the digestive tract through incisions, sutures, punctures and/or stapling, which can cause trauma to the organ being altered and lead to bleeding. For instance, bariatric surgery procedures can be used to treat obesity, and can be aimed at bypassing a portion of the stomach and/or the intestine. Such medical procedures can also lead to an increased risk of infection or other complications.

Magnetic compression anastomosis can be used in the context of medical procedures to treat conditions associated with the digestive tract. With magnetic compression anastomosis, necrosis is induced in tissue sandwiched between two magnets. A healing process takes place around the magnets, while the compressed tissue eventually dies and separates from surrounding living tissue. The magnets are released along with the necrotic tissue, leaving an open passage known as an anastomosis.

There remain a number of challenges with respect to surgery procedures in the digestive tract, particularly in the formation of an anastomosis.

### SUMMARY

In accordance with an aspect, there is provided A system for forming an anastomosis between two adjacent walls of a digestive tract, the system comprising:
first and second magnetic implants configured to magnetically couple to each other through the two adjacent walls of the digestive tract to compress a portion of the two adjacent walls therebetween and form a necrotic area that becomes surrounded by a scarred edge following a healing time period;
a positioning tool comprising:
   a positioning wand configured for laparoscopic positioning of at least one of the first and second magnetic implants, the positioning wand comprising:
      an elongated member sized and configured to have at least a portion thereof to be inserted into an abdominal cavity of a patient; and
      a distal tip provided at a distal end of the elongated member and comprising a guide magnet configured to magnetically couple with the at least one of the first and second magnetic implants through one of the two adjacent walls, the distal tip being configured to surf onto an outer surface of the one of the two adjacent walls and be moveable in response to a contact pressure upon contact therewith to translate the at least one of the first and second magnetic implants to a desired site of the anastomosis via the robotic arm;
a robotic positioning system comprising a robotic arm engageable with the positioning wand to impart movement on the distal tip to enable said surfing onto the outer surface of the one of the two adjacent walls.

In some implementations, the first and second magnetic implants are elongated magnetic implants.

In some implementations, the positioning tool comprise a plurality of positioning tools, and wherein the plurality of positioning tools comprises an endoscope or a delivery catheter, and the positioning wand.

In some implementations, the plurality of positioning tools further comprises a secondary laparoscopic grasper tool.

In some implementations, the robotic arm is configured for operation in at least five degrees of freedom.

In some implementations, the robotic arm comprises first and second robotic arms each comprising a corresponding positioning tool for delivery the first and second magnetic implants respectively.

In some implementations, the robotic arm comprises articulations to facilitate guiding the distal tip of the positioning wand of the magnetic implant along the digestive tract to the desired site of the anastomosis by surfing the distal tip onto the outer surface of the one of the two adjacent walls.

In some implementations, the distal tip is pivotally engaged with the elongated member via a pin, the distal tip forming a hinge pivoting back and forth about the pin.

In some implementations, the distal tip is pivotally engaged with the elongated member via a pin, the distal tip having a cylindrical shape with a central cylindrical longitudinal axis coinciding with the pin such that the distal tip is rotatable about the central cylindrical longitudinal axis.

In some implementations, the distal tip comprises a frame pivotally engaged with the distal end of the elongated member via a pin, the frame forming a hinge pivoting back and forth about the pin.

In some implementations, the distal tip comprises at least one cylindrical body pivotally engaged with the frame via a cylindrical body pin extending along a central cylindrical body longitudinal axis of a corresponding cylindrical body such that the cylindrical body is rotatable about the central cylindrical longitudinal axis.

In some implementations, the distal tip is engaged with the elongated member via a spherical swiveling joint.

In some implementations, the distal tip is engaged with the distal end of the elongated member via a biasable connection.

In accordance with another aspect, there is provided a method for forming an anastomosis between two adjacent walls of a digestive tract of a patient with a robotic positioning system, the method comprising:
robotically navigating a first magnetic implant into a first hollow organ of the digestive tract to a first location on one side of a desired anastomose site;
robotically navigating a second magnetic implant into a second hollow organ of the digestive tract to a second location on another side of the desired anastomose site;
robotically adjusting a positioning of at least one of the first and second magnetic implants, comprising:
   magnetically coupling, through one of the two adjacent walls, at least one of the first and second magnetic implants with a distal tip moveably engaged with a distal end of an elongated member of a positioning wand, the positioning wand being operatively engaged with a robotic arm of the robotic positioning system; and
   surfing the distal tip onto an outer surface of the one of two adjacent walls to translate the at least one of the first and second magnetic implants, the distal tip being movable relative to a distal end of the elongated member in response to a contact pressure upon contact with the outer surface of the one of two adjacent walls; and
magnetically coupling the first and second magnetic implants to each other through the two adjacent vessel walls of the digestive tract to compress a portion of the two adjacent walls therebetween and form a necrotic area during a healing time period.

In some implementations, robotically navigating at least one of the first and second magnetic implants comprises robotically controlling a positioning tool engaged with the robotic arm.

In some implementations, robotically navigating the at least one of the first and second magnetic implants comprises releasably engaging the first magnetic implant with the positioning tool.

In some implementations, the positioning tool comprises an endoscope or a delivery catheter.

In some implementations, robotically adjusting the positioning of the at least one of the first and second magnetic implants is performed according to at least five degrees of freedom.

In some implementations, surfing the distal tip onto the outer surface of the one of two adjacent walls comprises gliding the distal tip along the outer surface of the one of two adjacent walls.

In some implementations, surfing the distal tip onto the outer surface of the one of two adjacent walls comprises rolling the distal tip along the outer surface of the one of two adjacent walls.

In accordance with another aspect, there is provided a method for positioning at least one of first and second magnetic implants configured for forming an anastomosis at a target site between two adjacent walls of a digestive tract of a patient, the method comprising:
deploying the first magnetic implant into a first hollow organ lumen of the digestive tract;
deploying the second magnetic implant into a second hollow organ lumen of the digestive tract;
robotically inserting a distal tip and at least a portion of an elongated member of a positioning wand into an abdominal cavity of the patient, the distal tip being movably engaged with a distal end of the elongated member and comprising a guide magnet;
magnetically coupling the distal tip of the positioning wand with the second magnetic implant; and
robotically surfing the distal tip onto an outer surface of the second hollow organ while maintaining the magnetic coupling with the second magnetic implant to translate the second magnetic implant and bring the second magnetic implant in close proximity with the first magnetic implant to magnetically couple the first magnetic implant with the second magnetic implant.

In some implementations, robotically inserting the distal tip and the at least a portion of the elongated member of the positioning wand into the abdominal cavity of the patient is performed laparoscopically via a robotic arm.

In some implementations, the method further comprises releasably engaging the positioning wand with the robotic arm.

In some implementations, robotically displacing the distal tip of the positioning wand within the abdominal cavity is performed according to at least five degrees of freedom.

In some implementations, surfing the distal tip onto the outer surface of the one of two adjacent walls comprises gliding the distal tip along the outer surface of the one of two adjacent walls.

In some implementations, surfing the distal tip onto the outer surface of the one of two adjacent walls comprises rolling the distal tip along the outer surface of the one of two adjacent walls.

In accordance with another aspect, there is provided a system for forming an anastomosis between two adjacent walls of a digestive tract, the system comprising:
first and second magnetic implants configured to magnetically couple to each other through the two adjacent walls of the digestive tract to compress a portion of the two adjacent walls therebetween and form a necrotic area that becomes surrounded by a scarred edge following a healing time period; and
a robotic system configured for navigating at least one of the first and second magnetic implants to a site of the desired anastomosis, the robotic system comprising:
   a first external magnetic control assembly comprising a first magnetic field generation device configured to be placed on a first side of the patient;
   a second external magnetic control assembly comprising a second magnetic field generation device configured to be placed on a second side of the patient;
   the first and second external magnetic control assemblies being configured to provide power to move a position of the magnetic generation field means and magnetization direction with respect to the patient.

In some implementations, the first and second magnetic implants are elongated magnetic implants.

In some implementations, the elongated magnetic implants have an aspect ratio of about 1:2 to 1:40.

In accordance with another aspect, there is provided a method for navigating at least one of first and second magnetic implants configured for forming an anastomosis at a target site between two adjacent walls of a digestive tract of a patient, the method comprising:
introducing the at least one of first and second magnetic implants into a mouth of the patient;
robotically navigating the at least one of first and second magnetic implants to the target site using a robotic system external to the patient, the robotic system comprising:
   a first external magnetic control assembly comprising a first magnetic field generation device configured to be placed on a first side of the patient; and
   a second external magnetic control assembly comprising a second magnetic field generation device configured to be placed on a second side of the patient;
wherein robotically navigating the at least one of first and second magnetic implants to the target site comprises moving a position of at least one of the first and second magnetic field generation devices and magnetization direction with respect to the patient.

In some implementations, the first and second magnetic implants are elongated magnetic implants.

In some implementations, the elongated magnetic implants have an aspect ratio of about 1:2 to 1:40.

In accordance with another aspect, there is provided a robotic positioning system for positioning at least one of first and second magnetic implants configured for forming an anastomosis at a target site between two adjacent walls of a digestive tract of
a patient, the robotic positioning system comprising:
   a positioning wand comprising:
      an elongated member sized and configured to have at least a portion thereof to be inserted laparoscopically into an abdominal cavity of a patient; and
      a distal tip provided at a distal end of the elongated member and comprising a guide magnet configured to magnetically couple with the at least one of the first and second magnetic implants through one of the two adjacent walls, the distal tip being configured to surf onto an outer surface of the one of the two adjacent walls and be moveable in response to a contact pressure upon contact therewith to translate the at least one of the first and second magnetic implants to a desired site of the anastomosis via the robotic arm; and
a robotic arm engageable with the positioning wand to impart movement on the distal tip to enable said surfing onto the outer surface of the one of the two adjacent walls.

In some implementations, the first and second magnetic implants are elongated magnetic implants.

In some implementations, the elongated magnetic implants have an aspect ratio of about 1:2 to 1:40.

In some implementations, the robotic positioning system further comprises a plurality of positioning tools, and wherein the plurality of positioning tools comprises an endoscope or a delivery catheter, and the positioning wand.

In some implementations, the plurality of positioning tools further comprises a secondary laparoscopic grasper tool.

In some implementations, the robotic arm is configured for operation in at least five degrees of freedom.

In some implementations, the robotic arm comprises first and second robotic arms each comprising a corresponding positioning tool for delivery the first and second magnetic implants respectively.

In some implementations, the robotic arm comprises articulations to facilitate guiding the distal tip of the positioning wand of the magnetic implant along the digestive tract to the desired site of the anastomosis by surfing the distal tip onto the outer surface of the one of the two adjacent walls.

In some implementations, the distal tip is pivotally engaged with the elongated member via a pin, the distal tip forming a hinge pivoting back and forth about the pin.

In some implementations, the distal tip is pivotally engaged with the elongated member via a pin, the distal tip having a cylindrical shape with a central cylindrical longitudinal axis coinciding with the pin such that the distal tip is rotatable about the central cylindrical longitudinal axis.

In some implementations, the distal tip comprises a frame pivotally engaged with the distal end of the elongated member via a pin, the frame forming a hinge pivoting back and forth about the pin.

In some implementations, the distal tip comprises at least one cylindrical body pivotally engaged with the frame via a cylindrical body pin extending along a central cylindrical body longitudinal axis of a corresponding cylindrical body such that the cylindrical body is rotatable about the central cylindrical longitudinal axis.

In some implementations, the distal tip is engaged with the elongated member via a spherical swiveling joint.

In some implementations, the distal tip is engaged with the distal end of the elongated member via a biasable connection.

### BRIEF DESCRIPTION OF THE DRAWINGS

The attached figures illustrate various features, aspects and implementations of the technology described herein.

FIG. 1 is an exploded perspective view of a first elongated magnetic implant shown on one side of a desired site of an anastomosis and of a second elongated magnetic implant shown on another side of the desired site of the anastomosis, with a vessel wall of a first hollow organ and a vessel wall of a second hollow organ being shown therebetween, in accordance with an implementation.

FIG. 2 is a perspective view of the first and second elongated magnetic implants shown in FIG. 1, with the first elongated magnetic implant being shown in contact with the vessel wall of the first hollow organ and the second elongated magnetic implant being shown in contact with the vessel wall of the second hollow organ, at the desired site of the anastomosis.

FIG. 3 is a side view of the first and second magnetic implants shown in FIG. 2.

FIG. 4A is a perspective view of a magnetic implant and of a retention member, in accordance with an implementation.

FIG. 4B is a side view of the magnetic implant of FIG. 4A.

FIG. 5 is an exploded view of the magnetic implant of FIG. 4A.

FIG. 6 is a cross-sectional view of the magnetic implant of FIG. 4A.

FIG. 7 is a perspective view of a positioning wand that includes a distal tip, an elongated member and a handle.

FIG. 8 is an enlarged view of a portion of a positioning wand showing an example of a distal tip of the positioning wand.

FIG. 9 is an enlarged view of a portion of a positioning wand showing another example of a distal tip of the positioning wand.

FIG. 10 is an enlarged view of a portion of a positioning wand showing another example of a distal tip of the positioning wand.

FIG. 11 is an enlarged view of a portion of a positioning wand showing another example of a distal tip of the positioning wand.

FIG. 12 is an enlarged view of a portion of a positioning wand showing another example of a distal tip of the positioning wand.

FIG. 13 is an enlarged view of a portion of a positioning wand showing another example of a distal tip of the positioning wand.

FIG. 14 is a side view of a positioning wand and a magnetic implant, the positioning wand including an elongated member and a distal tip, a portion of the elongated member and the distal tip being shown in an abdominal cavity, and the magnetic implant being shown in a lumen of an organ of the digestive tract.

FIG. 15 is a side view of a positioning wand and a magnetic implant, the positioning wand including an elongated member and a distal tip including multiple segments, a portion of the elongated member and the distal tip being shown in an abdominal cavity, and the magnetic implant being shown in a lumen of an organ of the digestive tract.

FIG. 16 is a perspective view of a positioning wand that includes a distal tip and an adaptor.

FIG. 17 is a schematic representation of a robotic positioning system including a robotic arm operatively engaged with a positioning wand.

FIG. 18 is a schematic representation of an external robotic system that includes a first external magnetic control assembly comprising a first magnetic field generation device configured to be placed on a first side of the patient, and a second external magnetic control assembly comprising a second magnetic field generation device configured to be placed on a second side of the patient.

### DETAILED DESCRIPTION

Techniques described herein relate to robotically-assisted systems, devices and methods for delivering and/or positioning magnetic implants on either side of two adjacent walls of hollow structures of the digestive tract of a patient to form an anastomosis, in the context of procedures to treat various medical conditions associated with the digestive tract. The formation of the anastomosis can be achieved without puncturing the tissue of the hollow structures through which the anastomosis is formed, for example by robotically inserting a first magnetic implant into the lumen of a first hollow organ and a second magnetic implant into the lumen of a second hollow organ, robotically positioning the first and second magnetic implants at a desired anastomosis site, and by magnetically coupling the first and second magnetic implants together to compress the tissue of the adjacent walls therebetween. Compression of the wall tissue between the two magnetic implants results in a necrotic area that corresponds approximately to the surface area of the compression surface of the magnetic implant pair. Over time, the necrotic area becomes surrounded by an edge of scar tissue, or scarred edge. The formation of scar tissue can include collagen fiber deposition, neovascularization, and epithelial regeneration, and represents a dynamic equilibrium involving cells, their milieu, and the extracellular matrix. Cytokines secreted by platelets and inflammatory cells can promote the formation of new blood vessels and collagen synthesis which, in dynamic balance with collagen degradation, can contribute to determining the healing response. Two components of collagen are hydroxyproline and hydroxylysine, with hydroxyproline being synthesized under conditions of oxidative stress via the hydroxylation of proline, and being involved in the cellular transport of collagen. The synthesis and transport of wound collagen can thus be understood by monitoring the hydroxyproline content of the wound. The edge of scar tissue can thus be characterized by the fusion, or mechanical bonding, of the walls of each hollow organ through which the anastomosis is formed that occurs in part via fibrosis mechanisms. The scarred edge can thus form a fluid-tight seal around the anastomosis.

Proceeding with the robotic delivery of one of the first and second magnetic implants, or both, at the site of the desired anastomosis can contribute to increasing the accuracy and precision of such delivery, which can be performed both endoscopically and laparoscopically. Similarly, the robotic positioning of the first and second magnetic implants, or both, also contribute to increasing the accuracy and precision of the positioning of the magnetic implants and facilitate the magnetic coupling of the magnetic implants.

Various implementations and features of the robotically-assisted systems, devices and methods will now be described in greater detail in the following paragraphs.

### General Description of the System for Forming an Anastomosis

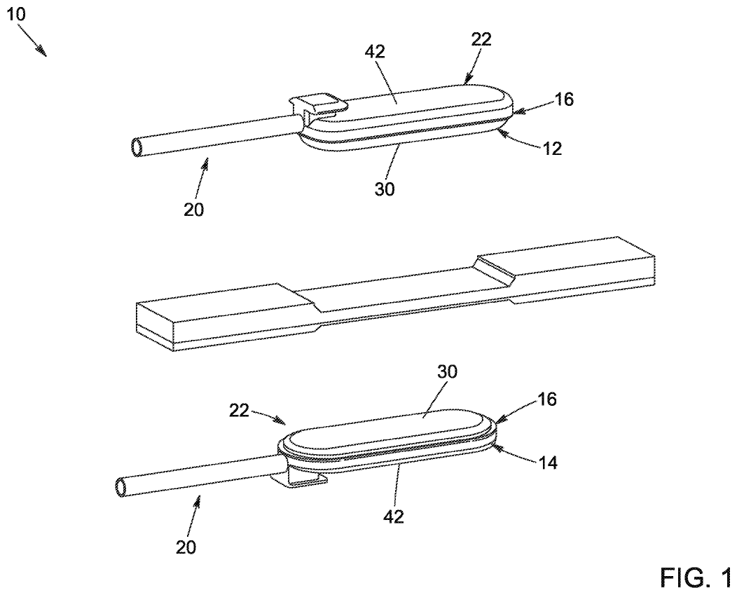

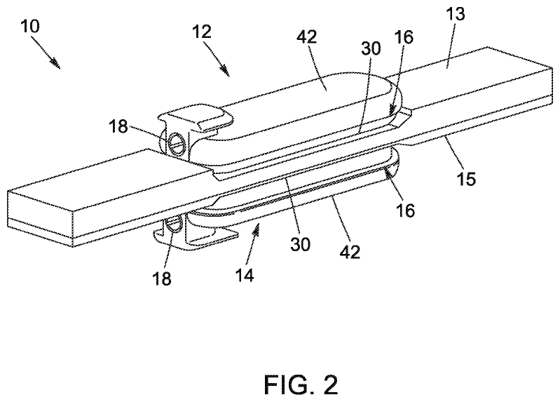

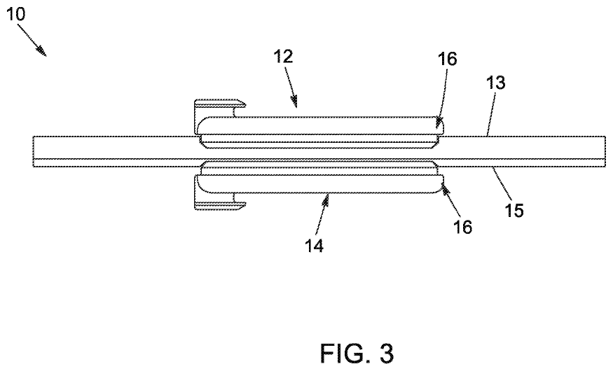

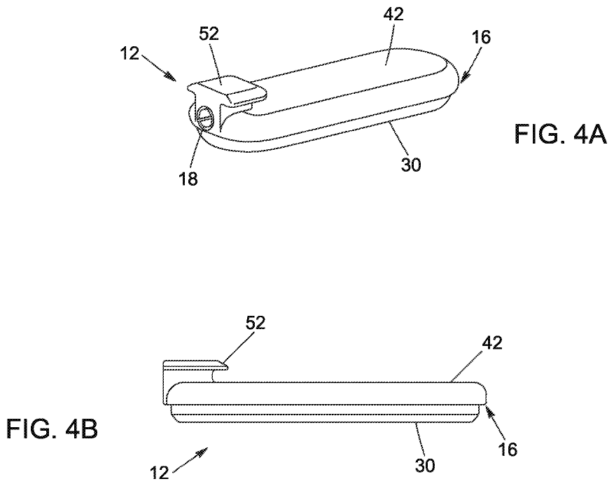

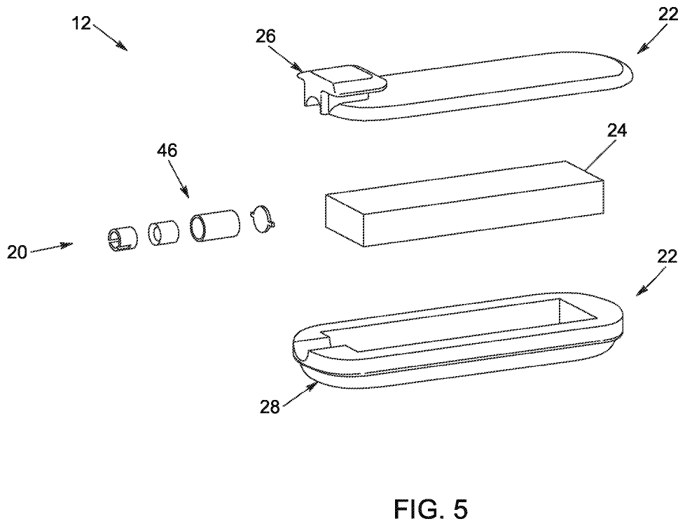

With reference to FIGS. 1 to 6, a system 10 for forming an anastomosis between two adjacent walls of hollow organs of the digestive tract is shown. Referring more particularly to FIG. 1, in the implementation shown, the system 10 includes a first magnetic implant 12 for implantation in the stomach, the first magnetic implant 12 being identified as a “stomach implant”; and a second magnetic implant 14 for implantation in the jejunum, the second magnetic implant 14 being identified as a “jejunum implant”. It is to be understood that the term “implant” refers to a device that is implanted in the digestive tract for a certain period of time, e.g., the healing time period, and that it can be used interchangeably with the term “device” or “component” for instance. In this implementation, the stomach represents a first hollow organ of the digestive tract into which the first magnetic implant 12 can be implanted, and the jejunum represents a second hollow organ into which the second magnetic implant 14 can be implanted, so as to compress a portion of the stomach wall 13 and a portion of the jejunum wall 15 therebetween.

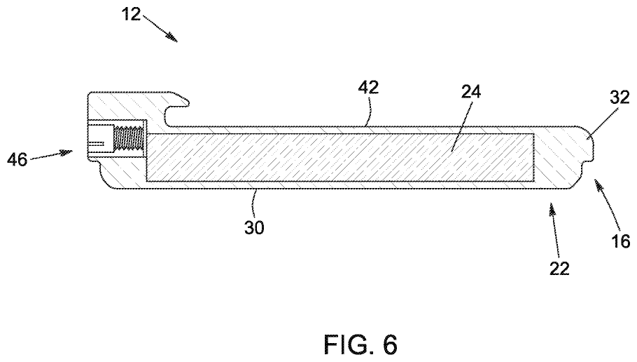

In FIGS. 1 to 6, each one of the first magnetic implant 12 and the second magnetic implant 14 is associated with a retention member 16, which is illustrated as corresponding to a flange 32 in FIG. 6. In the implementation shown, each one of the first magnetic implant 12 and the second magnetic implant 14 also includes a connecting member 18 that can be releasably engageable with a connector 20, which in FIG. 1 is identified as a delivery catheter. In other words, the magnetic implant 12, 14 can include a feature that enables its connection to a connector 20, which can be robotically manipulated, for navigating the magnetic implant 12, 14 to a desired site for creating the anastomosis. In turn, the connecting member 18 can include any feature that enables a releasable connection of the magnetic implant 12, 14 with the connector 20. In FIGS. 1-6, the connecting member 18 is shown as a “catheter attachment” that includes a catheter attachment assembly 46. The catheter attachment is configured as a receiving cavity that can receive a distal end of the connector 20 therein, which as mentioned above can be a robotically-assisted delivery catheter.

In some implementations and as shown in FIGS. 1 to 6, the magnetic implant 12, 14 can include a housing 22 that encloses a magnet 24 therein. The housing 22 can include for instance an outward portion 26 and an inward portion 28. The inward portion 28 includes the portion of the housing 22 that faces the corresponding other magnetic implant and is involved in the magnetic compression of the tissue, while the outward portion 26 of the housing 22 is on the opposed side of the magnetic implant facing away from the tissue being compressed. In this example, the inward portion 28 and the outward portion 26 surround the magnet 24 and can be coupled together around a periphery thereof. Other housing constructions are also possible, where one or more housing components are used to partly or fully enclose the magnet.

Each of these components of the system for forming an anastomosis will now be described in further detail.

### Description of the Magnetic Implant

Still referring to FIGS. 1 to 6, the first magnetic implant 12 is a device that is implantable into a first hollow organ of the digestive tract of a patient at a site of a desired anastomosis via the lumen of the first hollow organ. Examples of hollow organs of the digestive tract include the oesophagus, stomach, duodenum, jejunum, ileum, colon, biliary tract, and pancreatic duct. A site of desired anastomosis can be determined according to the condition of the patient, and this aspect will not be discussed further in the context of the present description. As used herein, the expression “magnetic implant” refers to a structure that can be implanted into the chosen hollow organ of the digestive tract, and that can be magnetically attracted to another magnetic implant due to magnetic forces. In some implementations, the magnetic implant can consist of a magnet. In some implementations, the magnetic implant can include a magnet and one or more additional features, such as a housing and/or a connecting member. The two magnetic implants can be substantially the same as each other, or different, in terms of their shape, configuration, construction, and/or material make-up. These features will be further discussed below.

The first magnetic implant 12 is used with a second magnetic implant 14 to form am implant pair. The second magnetic implant 14 is a device implantable into a second hollow organ of the digestive tract of the patient to the site of the desired anastomosis via the lumen of the second hollow organ. The second hollow organ of the digestive tract is located in sufficiently close proximity of the first hollow organ to enable the convergence of the respective wall tissue of the first hollow organ and the second hollow organ to eventually form the anastomosis.

The first and second magnetic implants 12, 14 are configured to remain within the digestive tract for at least a given healing time period. The healing time period enables necrosis of the anastomosis area while providing enough time for the edge of scar tissue to form. In some implementations, after approximately 3 to 5 days following implantation of the pair of magnetic implants at the desired site of the anastomosis, the periphery of the anastomosis is strengthened by collagen deposition, with the formation an edge of scar tissue having an increased tensile strength occurring at an estimated of approximately 7 to 10 days following implantation. The duration for forming the scar tissue can vary depending on the overall health of the individual patient, and depending on the specific parts of the digestive tract being joined. The scar tissue can also gain strength over the course of several additional weeks. In some implementations, it may be desirable for the magnetic implants to be released and passed out of the body of the patient about two weeks after implantation. In some implementations, the healing time period can be about two weeks, or at least two weeks.

Each one of the first and second magnetic implants 12 can be robotically navigated to the site of the desired anastomosis using various techniques. For instance, in some implementations, the magnetic implants 12, 14 can be delivered to the site of the desired anastomosis endoscopically using a robotic positioning system. In some implementations, at least one of the magnetic implants 12, 14 can be delivered laparoscopically using a robotic positioning system. These aspects will be discussed in further detail below.

Each one of the first and second magnetic implants 12, 14 can have any suitable shape and size determined in accordance with their intended purpose. In some implementations, the size and the shape of the magnetic implant can be determined for instance in accordance with the characteristics of the site of the desired anastomosis, the delivery technique chosen to deliver the magnetic implant to the site of the desired anastomosis, and so on. In some implementations, the magnetic implant can have for example an elliptic shape, a circular shape, an elongated shape, a rectangular shape, an octagonal shape, or any other polygonal shape in terms of its cross-section. The magnetic implant can include rounded corners to facilitate navigation into the digestive tract. The magnetic implant can have an aspect ratio of about 1:1 (e.g., in the case of a circular cross-section) or an aspect ratio of about 1:2 to 1:40, about 1:3 to 1:20, about 1:4 to 1:15, for example, or another aspect ratio. In some implementations, the shape and size of the retention member 16 can be adapted in accordance with the shape and size of a corresponding one of the first and second magnetic implants 12, 14. For instance, in some implementations, the height of the magnetic implant can be proportional to the thickness of the magnet 24 contained therein and hence desired magnetic strength.

Each of the first and second magnetic implants 12, 14 includes a compression surface 30 that is configured to contact the tissue of the corresponding hollow organ. The compression surface 30 can also be referred to as a tissue-contacting surface, since it is the surface of the magnetic implant that is eventually in contact with the interior wall of the hollow organ once the magnetic implant is delivered to the site of the desired anastomosis. Each of the first and second magnetic implants 12, 14 also includes a lumen-oriented surface 42 opposite the tissue-contacting surface, the lumen-oriented surface generally facing the lumen of the hollow organ once the magnetic implant is delivered to the site of the desired anastomosis.

In some implementations, the magnetic implant 12, 14 can include one or more magnets. The magnet 24 can be any type of suitable magnet composed of the appropriate material. In some implementations, the magnet 24 can be chosen according to its attractive force, i.e., according to the pressure that will be exerted on the surface area of the tissue that will eventually be compressed between the first and second magnetic implants 12, 14. Factors influencing the attractive force of the magnet 24 can include the shape of the magnet 24, the thickness of the magnet 24, the material of which the magnet 24 is made, etc. Example materials include neodymium magnets (e.g., NdFeB magnets), rare earth magnets, and ferrite magnets.

In some implementations, the magnet 24 or magnets of a first magnetic implant 12 may be made of a magnetic material that is not permanently magnetized, such as soft magnetic alloys, e.g., nickel-iron, silicon iron, iron, iron-cobalt, and ferritic stainless steels. In other words, the magnet(s) of respective magnetic implants may not be constructed of two permanent magnets. In other implementations, the magnets 24 of a first and second magnetic implants 12, 24 may be constructed of two permanent magnets.

In addition, the first and second magnetic implants 12, 14 can include one or more features described in granted U.S. Pat. Nos. 11,576,676, 11,534,171 and 11,583,280, which are incorporated herein by reference in their entirety.

### Delivery and Positioning of the Magnetic Implants in the Digestive Tract

Various robotic techniques can be implemented to deliver, navigate and/or guide the magnetic implants 12, 14 to the site of the desired anastomosis, and/or to positioning the magnetic implants 12, 14 at the site of the desired anastomosis, i.e., at a target site. As mentioned above, proceeding with the robotic delivery of one of the first and second magnetic implants 12, 14, or both, at the site of the desired anastomosis can contribute to increasing the accuracy and precision of such delivery, which can be performed both endoscopically and laparoscopically. Similarly, the robotic positioning of the first and second magnetic implants 12, 14, or both, also contribute to increasing the accuracy and precision of the positioning of the magnetic implants, and can enable micropositioning of the magnetic implants 12, 14.

In robotically-assisted or telerobotic surgery, the surgeon typically operates a master controller to remotely control the motion of surgical instruments at the surgical site from a location that may be remote from the patient (e.g., across the operating room, in a different room or a completely different building from the patient). The master controller usually includes one or more hand input devices, such as joysticks, exoskeletal gloves or the like, which are coupled to the surgical instruments with servo motors for articulating the instruments at the surgical site. The servo motors are typically part of an electromechanical device or surgical manipulator (“the slave”) that supports and controls the surgical instruments that have been introduced directly into an open surgical site or through trocar sleeves into a body cavity, such as the patient's abdomen. During the operation, the surgical manipulator provides mechanical articulation and control of a variety of surgical instruments, such as tissue graspers, needle drivers, electrosurgical cautery probes, etc., that each performs various functions for the surgeon, e.g., holding or driving a needle, grasping a blood vessel, or dissecting, cauterizing or coagulating tissue.

The number of degrees of freedom (DOFs) is the number of independent variables that uniquely identify the pose/configuration of a robotic positioning system. Since robotic manipulators are kinematic chains that map the (input) joint space into the (output) Cartesian space, the notion of DOF can be expressed in any of these two spaces. In particular, the set of joint DOFs is the set of joint variables for all the independently controlled joints. Without loss of generality, joints are mechanisms that provide, e.g., a single translational (prismatic joints) or rotational (revolute joints) DOF. Any mechanism that provides more than one DOF motion is considered, from a kinematic modeling perspective, as two or more separate joints. The set of Cartesian DOFs is usually represented by the three translational (position) variables (e.g., surge, heave, sway) and by the three rotational (orientation) variables (e.g., Euler angles or roll/pitch/yaw angles) that describe the position and orientation of an end effector (or tip) frame with respect to a given reference Cartesian frame.

Telerobotic surgery through remote manipulation can improve the accuracy and precision of endoscopic and laparoscopic manipulations made by a surgeon, which in turn can contribute to reducing the overall time taken to perform the medical procedure and helping to reduce patient trauma and discomfort.

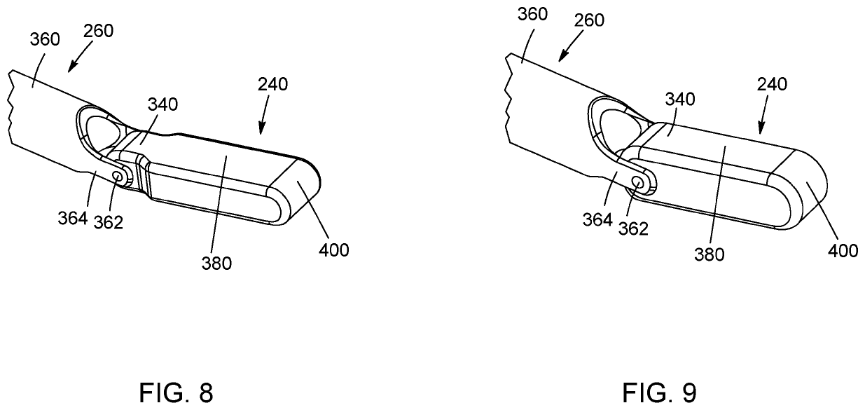

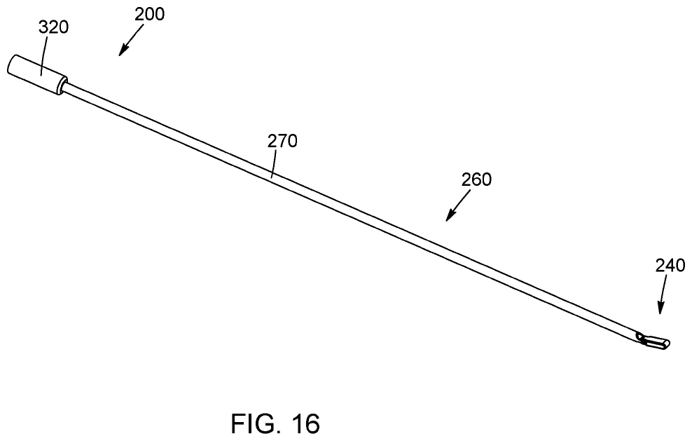

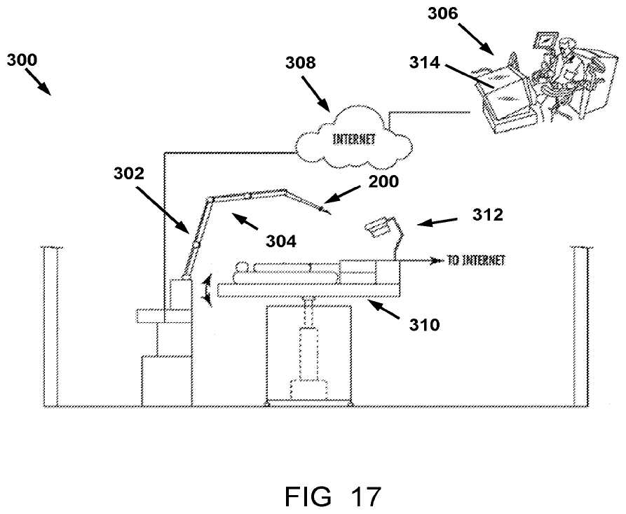

FIG. 17 illustrates a robotic positioning system 300 (shown in FIG. 8) configured for use in cooperation with a positioning wand 200 (shown in FIG. 16). The robotic positioning system 300 includes a robotic surgery station 302 having a robotic arm 304, and a remote surgeon station 306 that can be at another location from the robotic surgery station 302. A communications network 308, such as the Internet, couples the robotic surgery station 302 and the remote surgeon station 306. The robotic surgery station 302 can be positioned adjacent the operating table 310 and has at least one positioning tool, which can be the positioning wand 200, or another type of positioning tool, such as an endoscope or a catheter. A camera 312 can be located at the robotic surgery station 302 and the remote surgeon station 306 can include at least one display 314 coupled to the camera 312 via the communications network 308. Certain setup and active joints and links in the robotic arm 304 may be omitted to reduce the robot's size and shape, or joints and links may be added to increase degrees of freedom. It should be understood that the robotic arm 304 may include various combinations of links, passive joints, and active joints (redundant DOFs may be provided) to achieve a necessary range of motion for the delivery and positioning the magnetic implants.

The robotic positioning system 300 can include one or more features described for instance in granted U.S. Pat. Nos. 11,517,379, 10,856,946 and 10,813,710, which are incorporated herein by reference in their entirety. It is to be understood that other types of surgical robots as known in the art can also be used in combination with the positioning wand, an endoscope or a delivery catheter for assisting in the delivery, navigation, guiding and/or positioning of the magnetic implants. Examples of commercially available surgical robots within the scope of the present description include for instance the Da Vinci surgical system™, commercially available from Intuitive Surgical™, the Flex Robotic System™ from Novus™, surgical robots from Moon Surgical™, the Hugo™ RAS robotic system from Medtronic™, the surgical robot from Avatera™, the Revo™ and Revo-i™ robotic surgical systems from Cambridge Medical Robotics Surgical™ and revosurgical™, the Hinotori™ surgical robot system from Medicaroid™, the Senhance® surgical system from Asensus Surgical™, the Dexter™ surgical robot from Distalmotion™, the Flex® robotic system from Robotics Surgical™, the Vicarious Surgical™ robotic system from Vicarious Surgical™, and the MARS™ surgical system from Levita™, etc. It is to be understood that the present application is not limited to a particular robotic surgical system, and that the concepts described herein can be put in application with any suitable robotic surgical system.

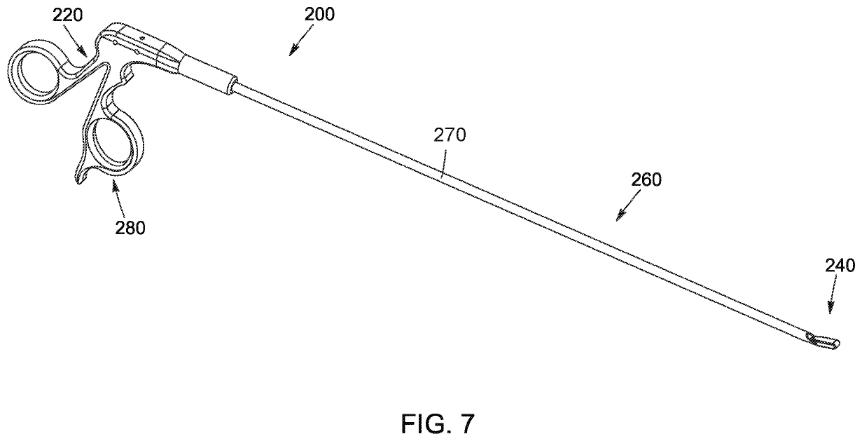

Referring more particularly to FIGS. 7 and 16, an implementation of a positioning wand 200 is shown. The positioning wand 200 includes a distal tip 240 at a distal end thereof, and an elongated member 260 extending between the handle 220 and the distal tip 240. The combination of the handle 220, elongated member 260 and distal tip 240 enables interaction with the magnetic implant within the abdominal cavity of the patient. In the implementation shown in FIG. 7, the positioning wand 200 includes a handle 220 at a proximal end thereof, whereas in the implementation shown in FIG. 16, the positioning wand 200 includes an adaptor 320 at a proximal end thereof. It is to be noted that in the context of the present description, the expression “positioning wand” can be used interchangeably with the expression “positioning device”.

Components of the positioning wand 200 will now be described in further detail in the following paragraphs. It is to be understand that the positioning wand 20 can include one or more of the components described below, and that there can be substitutions between such components to achieve a desired configuration of the positioning wand 200. Accordingly, although each combination of components is not explicitly described in detail in the present application, it is to be understood that the positioning wand 200 can include any one of the components as described herein, or a combination thereof.

### Elongated Member

The elongated member 260 can have various configurations. In some implementations, the elongated member 260 can include one or more tubular structures provided in a longitudinally adjacent relationship to provide a desired length of the elongated member 260. The tubular structure can be rigid or flexible. Accordingly, the elongated member 260 can include a single tubular structure that is rigid or flexible, or the elongated member 260 can include a plurality of tubular structures that are flexible or rigid, or can include a combination of tubular structures that are flexible and of tubular structures that are rigid. In some implementations, when the elongated member 260 includes a plurality of tubular structures, the tubular structures can be rigid, and the connection between adjacent tubular structures can be such that the resulting entire length of the elongated member 260 is rigid. Alternatively, when the elongated member 260 includes a plurality of tubular structures, the connection between two adjacent tubular structures can be such that one of the tubular structures can move relative to the adjacent tubular structure, thereby conferring flexibility to at least a portion of the elongated member 260. For instance, enabling movement of a distal one of the tubular structures relative to a proximal one of the tubular structures can facilitate navigating the elongated member 260 to a desired location within the abdominal cavity of the patient. When the connection between two adjacent tubular structures is flexible, the flexibility of the connection can be provided for instance by a material that is more flexible than the material from which is made the tubular structure. For example, the flexible connection can be made of a polymer such as silicone. In some implementations, when the elongated member 260 includes a plurality of tubular structures, the plurality of tubular structures can be provided in a telescopic relationship to form a telescopic assembly of tubular structures. The telescopic assembly of tubular structures can enable adjusting the length of the elongated member 260 so that the elongated member 260 can be switched from a deployed configuration when a given length of the elongated member 260 is desired, for instance to reach a certain location within the abdominal cavity, to a retracted configuration when a shorter length of the elongated member 260 is desired or for storage of the positioning wand 200.

The tubular structure forming the elongated member 260 can be hollow and define a channel extending along a longitudinal axis thereof. When a plurality of tubular structures is provided, the hollow configuration of the tubular structure can enable the elongated member 260 to adopt the telescopic configuration described above. The hollow configuration of the tubular structure can enable the passage of one or more pull wires, or guide wires, therein. In such implementations, the hollow tubular structure thus serves as a housing for receiving the one or more pull wires. The presence of a pull wire within the channel of the tubular structure can contribute to facilitating steering of the elongated member 260, when the elongated member 260 is made of at least one tubular structure that is flexible or that includes a plurality of the tubular structures that are flexibly connected with each other. Once again, being able to steer the elongated member 260 via the action of the pull wire can be beneficial to deploy the positioning wand 200 to a desired location within the abdominal cavity of the patient.

In the implementation shown in FIG. 7, the elongated member 26 includes a single tubular structure 270 that is substantially rigid.

The dimensions of the elongated member 260 can vary depending on the intended application or maneuver for which the positioning wand 200 is to be used for. In some implementations, the elongated member 260 can be configured to fit through a laparoscopic trocar having an internal diameter ranging from about 3 mm to about 15 mm. Accordingly, in such implementations, the external diameter of the elongated member 26 can range for instance from about 2 mm to about 14 mm.

The elongated member 260 can also be configured to enter the abdominal cavity through a NOTES procedure. In such implementations, the elongated member 260 is generally flexible to facilitate navigation thereof through the sinuous pathway of the digestive tract. A NOTES procedure is a procedure that involves gaining access to the abdominal cavity by entering the digestive tract through a natural orifice rather than percutaneously. The natural orifice can vary depending on the location that is to be reached, and can include the mouth, the anus, or the vagina. Access via the mouth can enable a distal portion of the elongated member 260 to enter the stomach with the option of travelling further down in the digestive tract towards the small intestine, similarly to how an endoscope would be used, while access via the anus can enable a distal portion of the elongated member 260 to enter the colon and travel up towards the small intestine, similarly to how a colonoscope would be used. The wall of the digestive tract can then be breached to enable passage of the elongated member 260 therethrough such that the elongated member 260 can enter the abdominal cavity at a chosen location. When the NOTES procedure is performed via the mouth and the incision is made through the wall of the stomach, the procedure can be referred to as a transgrastic NOTES procedure. The incision made through the wall of the digestive tract can then be sutured using endoscopically administered clips, for instance. A NOTES procedure can thus avoid an incision of the abdominal wall of the patient, which can also avoid complications that can occur from abdominal wall incisions, such as hernias or wound infections. In some implementations, the use of an elongated member 260 that is flexible can facilitate access to remote areas of the peritoneal cavity more easily and quickly compared to a rigid elongated member 260.

Thus, it will be understood that the characteristics of the elongated member 260, for instance in terms of dimensions and properties, can be determined and adapted in accordance with the intended use of the positioning wand 200 and more particularly, on the location of the desired site of the magnetic compression anastomosis and the maneuvers that have to be performed in the abdominal cavity to access the desired site of the magnetic compression anastomosis.

### Distal Tip

Details regarding the engagement of the distal tip 240 with the elongated member 260 and the configuration of the distal tip 240 will now be provided.

The distal tip 240 of the positioning wand 200 is configured to magnetically interact with at least one of the magnetic implants 12, 14, to aid in the placement of the magnetic implants 12, 14 at the desired site of the anastomosis. Once a magnetic interaction of the distal tip 240 with a magnetic implant has been established, the health care provider can move the distal tip 24 via interaction with the robotic arm 302 to bring the magnetic implant at the desired site of the anastomosis. Moving the magnetic implant via a magnetic interaction with the distal tip 240 can involve for instance lightly sliding the distal tip 240 along the outer surface of the wall of an organ of the digestive tract (which can also be referred to as “surfing” or “gliding” the distal tip 240 along the wall of an organ of the digestive tract), such as the small intestine, or rotating or rolling the distal tip 240 along the wall of an organ of the digestive tract. The distal tip 240 can also be configured to be moveable in response to a contact pressure upon contact with the wall of the digestive tract to lighten the contact pressure, to minimize tissue trauma to the wall of the organ of the digestive tract. It is to be understood that as used herein, the expressions “surfing” and “gliding” can be used interchangeably, and designate the light contact between the distal tip of the positioning wand and the outer surface of the wall of the organ of the digestive tract, as described in further detail below.

The surfing, or gliding, of the distal tip 240 of the positioning wand 200 onto the outer surface of the wall of the digestive tract can contribute to provide an improved delivery technique compared to other techniques known in the art which involve for instance “milking” the large intestine or the small intestine. Intestinal milking can also be referred to as “stripping” or “handling decompression”. When such technique is used, the intestine is grasped and squeezed above or below material occupying the lumen of the intestine, for instance when compaction with faeces has occurred, and the squeeze is maintained and moved along the digestive tract to in turn actively move, or “milk”, this material upstream or downstream in the digestive tract. The squeezing can be performed using a pincer-like instrument configured to pinch the intestine substantially perpendicularly to the direction of flow. However, this technique has several drawbacks, including compromising the physical integrity of the wall of the digestive tract, formation of peritoneal adhesion, damage to visceral peritoneum on the intestinal wall, etc. These drawbacks are often the result of the friction imparted to the wall of the intestine by the tool, e.g., pincher, or fingers imparting the milking, the pressure trauma exerted by the tool or fingers imparting the milking, and/or the stretching trauma exerted by the tool or fingers imparting the milking. This type of technique is therefore not desirable. In contrast, the “surfing” technique described herein eliminates these drawbacks, given that the intestine is not squeezed during the procedure, but the magnetic implant is rather magnetically coupled with a positioning tool such as a positioning wand, with the magnetic implant being located within the lumen of the hollow organ of the digestive tract while the distal tip is located outside of the hollow organ, such that the wall of the hollow organ is located between the magnetic implant and the distal tip of the positioning wand. With such a magnetic coupling, and as described above, the distal tip of the positioning wand can be lightly surfed onto the outer surface of the wall of the organ of the digestive tract, and can be moveable in response to a contact pressure upon contact with the wall of the digestive tract to lighten the contact pressure, so as to minimize tissue trauma to the wall of the organ of the digestive tract. The terms “skimming”, “wafting” and “hovering” are also considered within the intended scope of the term “surfing” to designate the light contact between the distal tip of the positioning wand and the outer surface of the wall of the organ of the digestive tract, in contrast with “milking”.

With reference to FIGS. 8 to 15, the distal tip 240 of the positioning wand 200 can have various shapes and configurations. For instance, the distal tip 240 can have an elongated shape, a rectangular shape, a cylindrical shape, an oblong or stadium shape, an elliptic shape, a “pill” shape, or a wedged shaped. The distal tip 240 can include atraumatic edges to facilitate insertion and navigation into the digestive tract and the abdominal cavity. When referring to a rectangular shape, it is to be understood that the cross-section of the distal tip 240 can be rectangular, and the distal tip 240 can have the shape of a rectangular prism.

In some implementations, the distal tip 240 can have a length ranging from about 3 mm to about 60 mm, and a width ranging from about 2 mm to about 14 mm. In some implementations, the distal tip 240 can have a length of up to 100 mm. When a laparoscopic trocar is used to introduce the positioning wand 20 into the abdominal cavity of the patient, the width of the distal tip 240 can be determined so as to fit within to opening formed by the laparoscopic trocar. The combination of the length and width of the distal tip 240 can be determined to achieve a given surface area of the distal tip 240 to efficiently interact with the magnetic implant, and accordingly to achieve a given magnetic load of the distal tip 240. For instance, it may be desired to achieve a given surface area of the distal tip 240 that enables distributing the magnetic load over such given surface area to enable “surfing” onto the outer surface of the wall of the digestive tract and thus minimize tissue trauma to the wall of the organ of the digestive tract. In some scenarios, a larger surface area of the distal tip 240 can contribute to minimizing tissue trauma to the wall of the organ of the digestive tract.

FIGS. 8 and 9 illustrate an implementation where the distal tip 240 has an oblong shape that is elongated along a longitudinal axis of the elongated member 260, and that includes rounded corners and edges to facilitate navigation into the digestive tract and/or the abdominal cavity. In the illustrated implementations shown in FIGS. 8 and 9, a proximal portion 340 of the distal tip 240 is pivotally engaged with the distal end 360 of the elongated member 260, and the remainder of the body 380 of the distal tip 240, including the distal portion 400 of the distal tip 240, extends longitudinally from the distal end 360 of the elongated member 260. The pivotable engagement can be achieved via a pin 362 coupled to a hinge 364. Multiple pin and hinge couplings can be provided in parallel, to provide multiple in-line pivotable engagements. In some implementations, multiple pins may be oriented orthogonally, to create one or more universal joints as the pivotable engagement. The pivotal engagement enables the distal tip 240 to pivot back and forth about the pin 362.

The pivotable engagement of the proximal end 340 of the distal tip 240 with the distal end 360 of the elongated member 260 can enable the distal tip 240 to rotate around a rotation axis that is perpendicular to the longitudinal axis of the elongated member 26. For instance, in FIGS. 8 and 9, the distal tip 240 could be considered as being configured to move up and down in relation to the distal end 360 of the elongated member 260, although the distal tip 240 could also be considered as being configured to move side to side in relation to the distal end 360 of the elongated member 260 following a rotation of 90°of the elongated member 260. Thus, the distal tip 240 of the positioning wand 200 can move in several directions when the elongated member 260 is manipulated to be rotated around its longitudinal axis.

FIG. 8 illustrates a distal tip 240 having a proximal portion 340 that has a reduced width compared to the remainder of the body 380 of the distal tip 240. In some implementations, the reduced width of the proximal portion 340 of the distal tip 240 can facilitate the cooperation with the distal end 360 of the elongated member 260. The variation of the width of the distal tip 240 along a length thereof can also enable adapting the width of the proximal portion 340 of the distal tip 240 to the diameter, or width, of the elongated member 260. For instance, it may be desired that the distal tip 240 has a given width that is larger than the diameter of the elongated member 260 at the distal end 360 thereof, in which case the width of the proximal portion 340 of the distal tip 240 can be reduced to facilitate cooperation with the distal end 360 of the elongated member 260. Alternatively and as shown in FIG. 9, the width of the distal tip 240 can remain substantially the same throughout its length, and can thus have a width similar to the width of the distal end 360 of the elongated member 260.

In some implementations, the pivotable engagement of the distal tip 240 with the distal end 360 of the elongated member 260 can facilitate the magnetic interaction of the distal tip 240 with a magnetic implant, by enabling the distal tip 240 to deviate from the longitudinal axis of the elongated member 260 to get closer to the wall of the organ of the digestive tract. The pivotable engagement of the distal tip 240 with the distal end 360 of the elongated member 260 can also enable movement of the distal tip 240 relative to the elongated member 260 so that the distal tip 240 can adjust and/or conform to the variability in the outer surface of the small intestine, for instance, or another organ of the digestive tract, thus enabling “surfing” onto the outer wall of the organ of the digestive tract.

In both FIGS. 8 and 9, the distal tip 240 includes a distal portion 400 that has rounded corners to give the oblong shape to the distal tip 240. The rounded corners can contribute to avoiding traumatic contact with the wall of the digestive tract when the distal tip 240 is navigated within the digestive tract, and when the distal tip 240 is navigated within the abdominal cavity of the patient.

The length of the distal tip 240 can also vary, and can be influenced for instance by the desired range of motion of the distal tip 240. For instance, the longer the length of the distal tip 240, the longer the arc length can be, thus enabling a wider range of motion of the distal tip 240.

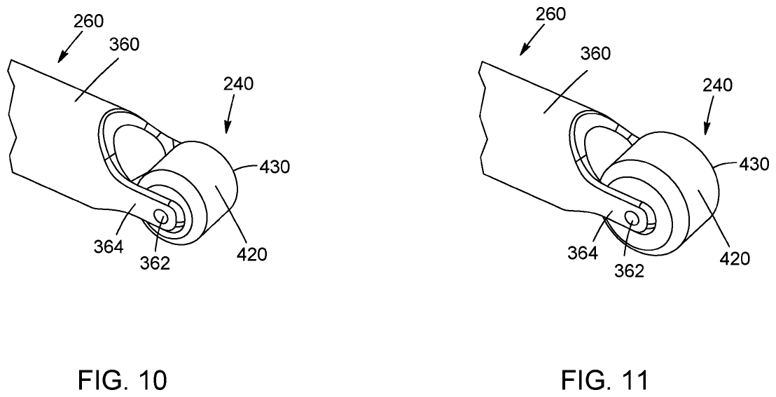

In other implementations, the distal tip 240 can have a cylindrical shape such as shown in FIGS. 10 to 12. In the implementations shown in FIGS. 10 and 11, the distal tip 240 has a cylindrical shape with a longitudinal axis, or central axis, that extends substantially perpendicular to the longitudinal axis of the elongated member 260. The distal tip 240 is pivotally engaged with the distal end 36 of the elongated member 260 via its central axis, such that the distal tip 240 can rotate up to 360° relative to the pivot axis. Accordingly, in such implementations, the distal tip 240 can be viewed as a wheel-type distal tip, and the distal tip 240 can be defined as being pivotally engaged with the elongated member 260, or rotatably engaged with the elongated member 260. A wheel-type distal tip can contribute to facilitating insertion and navigating into the digestive tract and abdominal cavity by enabling the outer surface 420 of the cylinder 430 to roll, or rotate, against the outer wall of the organ of the digestive tract or against various surfaces in the abdominal cavity, thereby reducing friction between the outer surface 420 of the cylinder 430 and the outer wall of the organ of the digestive tract or the various surfaces in the abdominal cavity, and reducing the sliding motion of the distal tip 240 against the outer wall of the organ of the digestive tract. Thus, in implementations where the distal tip 240 comprises a wheel-type distal tip, the surface of the wheel can roll onto the wall of the organ of the digestive tract, instead of sliding against it. As is illustrated in FIGS. 10 and 11, the diameter of the distal tip 240, when the distal tip 240 is shaped as a wheel-type distal tip, can vary and can be adapted in accordance with the intended application of the positioning wand 200.

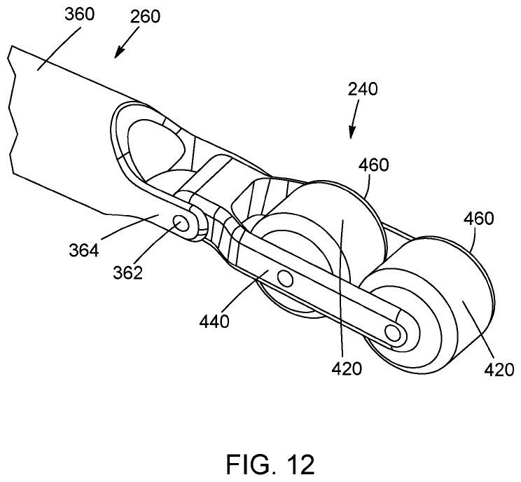

FIG. 12 illustrates another exemplary implementation of the distal tip 240. In the implementation shown, the distal tip 240 includes a frame 440 pivotally engaged with the distal end 360 of the elongated member 260 via the pin 362 and hinge 364 described above. The frame 440 is configured to receive two cylindrical bodies 460, each one of the cylindrical bodies 460 being pivotally engaged with the frame 440. In this implementation, the frame 440 can thus rotate relatively to the elongated member 260, similarly to the distal tip 240 illustrated in FIGS. 10 and 11, and the cylindrical bodies 460 can rotate relative to the frame 440. This configuration of the distal tip 240 enables the distal tip 240 to have a range of motion along the arc length resulting from the rotation of the frame 440 relative to the elongated member 260, and can reduce friction between the respective outer surfaces 420 of the cylindrical bodies 460 and the outer wall of the organ of the digestive tract or the various surfaces in the abdominal cavity.

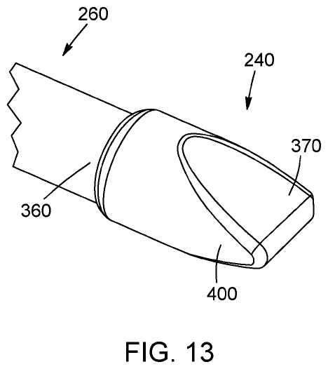

FIG. 13 illustrates yet another exemplary implementation of a distal tip 240. In this implementation, the distal tip 240 has a wedge shape and is fixedly engaged with the distal end 360 of the elongated member 260. In FIG. 13, the wedged portion 370 is provided at a distal end 400 of the distal tip 240. The wedge shape of the distal tip 240 can provide a wider surface area for the distal tip 240 to slid along the wall of the digestive tract, thereby reducing potential trauma to the tissues, such as the wall of the bowel.

In some implementations, the distal tip 240 can be engaged with the elongated member 260 via a spherical swiveling joint. In some implementations, the distal tip 240 can be engaged with the distal end of the elongated member 260 via a biasable connection. The biasable connection can include for instance a spring, or a biasable neck extending between the distal end 360 of the elongated member 260 and the distal tip 240.

In addition, the positioning wand 200 can include one or more features described in granted U.S. Pat. No. 11,534,171, which is incorporated herein by reference in its entirety.

Thus, in order to have the distal tip 240 of the positioning wand 200 lightly surfed onto the outer surface of the wall of the organ of the digestive tract, the distal tip 240 can be glided onto the outer surface of the wall of the organ of the digestive tract (for instance when the distal tip 240 has a configuration as shown in FIGS. 8 and 9), or rolled onto the outer surface of the wall of the organ of the digestive tract (for instance when the distal tip 240 has a configuration as shown in FIGS. 10 to 12), among other techniques.

As mentioned above, the positioning wand 200, rather than including a handle 220 for manual handling by a healthcare provider such as shown in FIG. 7, can include an adaptor 320 to enable coupling with the robotic arm 302 of the robotic surgery station 300, as schematically illustrated in FIG. 16. The handle 220 can thus be removed or omitted, and the positioning wand 200 can include only the adaptor 320. Any type of adaptor 320 that enables such coupling with the robotic arm 302 as known in the art can be suitable. Alternatively to the positioning wand 200, laparoscopic instruments and devices can be found described in U.S. Pat. No. 11,607,223 and/or PCT/US2021/072886, which are incorporated herein by reference in their entirety, and are also within the scope of the present description. Thus, the positioning wand 200 can have structures enabling manual use, as illustrated, or can have a connection member or an adaptor 320 that enables attachment, or coupling, to the robotic arm 302. The positioning wand 200 can be removably attached to the distal end of the robotic arm 302 for removal, maintenance, cleaning and the like.

The positioning of at least one of the magnetic implants at the desired site of the anastomosis can be performed robotically using the robotic positioning system 300 via the positioning wand 200 or another laparoscopic instrument, including non-magnetic instruments, interacting with the magnetic implant within the abdominal cavity of the patient. The positioning wand 200 can be manipulated robotically by a healthcare provider, such as a physician, and a portion of the positioning wand can be robotically introduced into the abdominal cavity of a patient using a minimally invasive surgery. Minimally invasive surgeries can include laparoscopic surgeries, which typically includes cooperation of a laparoscopic instrument with a trocar to facilitate introduction of the laparoscopic instrument into the abdominal cavity, percutaneous laparoscopy, which can enable introduction into the abdominal cavity without the use of a trocar, and Natural Orifice Transluminal Endoscopic Surgery (NOTES) procedures, for example.

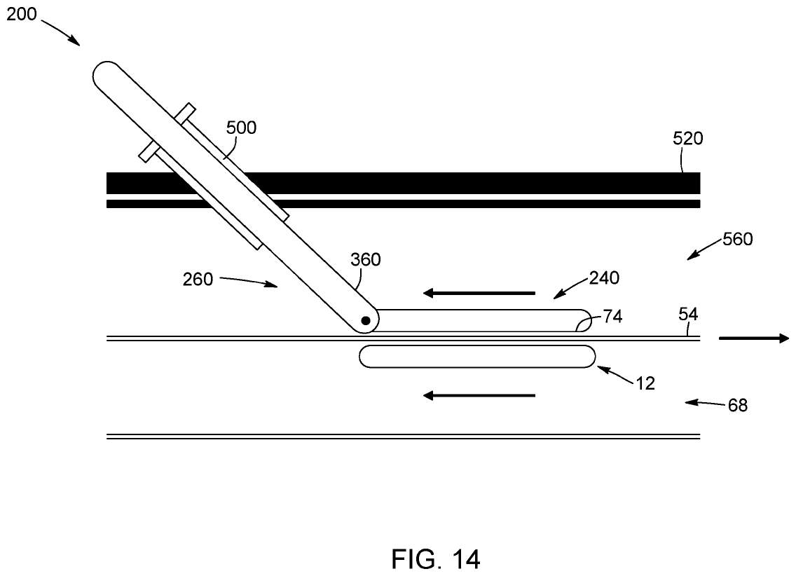

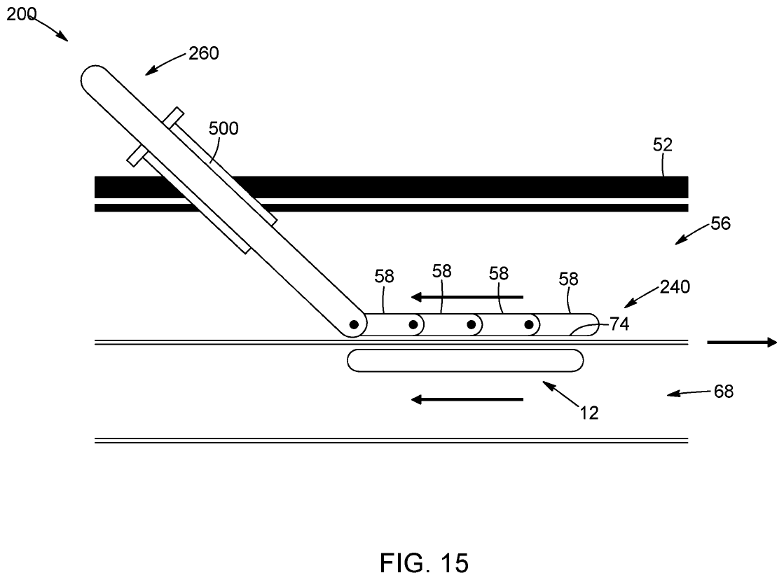

With reference to FIGS. 14 and 15, in implementations where the positioning tool includes a positioning wand 200, a laparoscopic trocar 500 can be inserted through the abdominal wall 520 of a patient, with the elongate member 260 of the positioning wand 200 being inserted through the opening of the laparoscopic trocar 500 and into the abdominal cavity 560. The positioning wand 200 further includes a distal tip 240 provided at the distal end 360 of the elongated member 260. The distal tip 240 can be hingedly connected to the distal end 360 of the elongate member 260, for instance via a hinge pin, although various alternatives are within the scope of the present description. The distal tip 240 can be configured to conform to an outer surface of the wall of a body lumen, i.e., the outer surface 54 of an organ of the digestive tract. The magnetic implant 12 is positioned within the lumen 68 of the organ of the digestive tract, such as the lumen of the bowel. The distal tip 240 can be magnetically attracted to the magnetic implant 12 through the wall of the organ of the digestive tract. The shape and material selection of the distal tip 240 are designed such that the magnetic attraction force between the distal tip 240 and the magnetic implant 12 is sufficient to enable robotically translating, or dragging, the magnetic implant 12 within the lumen of the organ of the digestive tract while minimizing the pressure on the outer surface of the wall of the organ of the digestive tract, to prevent damage to the wall of the organ. The magnetic implant 12 is thus translated within the bowel by robotically moving the distal tip 240 relative to the outer surface of the wall of the organ of the digestive tract. As mentioned above, robotically moving the distal tip 240 relative to the outer surface of the wall of the organ of the digestive tract can be done by surfing the distal tip 240 onto the outer wall of the organ of the digestive tract to enable the translation or dragging of the magnetic implant within the lumen of the organ of the digestive tract.

Referring now to FIG. 15, there is shown an implementation of the distal tip 240 that includes multiple segments 58 that are hingedly connected via hinge pins, as well as being hingedly connected to the distal end 360 of the elongate member 260. In this implementation, the assembly of multiples segments 58 forms a flexible train, or chain, of distal tip elements that can be conformable to the outer surface of the wall 54 of the organ of the digestive tract, while being configurable at an angle relative to the longitudinal axis of the elongate member 260.

In some implementations, the organ into which is received the magnetic implant 12 can be held in position with a secondary laparoscopic grasper tool, which can be controlled robotically, while the distal tip 240 is being robotically translated in the opposite direction. Alternatively, the magnetic implant 12 can be robotically translated by fixing the distal tip 240 and by robotically translating the organ with the secondary laparoscopic grasper tool. Thus, when the secondary laparoscopic grasper tool is used, the robotic positioning system 300 can include an additional robotic arm configured to robotically control the secondary laparoscopic grasper tool.

In other implementations, the positioning tool can be an endoscope or a catheter, such as a delivery catheter. In such implementations, the endoscope or catheter includes an adaptor 320 to enable coupling with the robotic arm 302 of the robotic surgery station 300. Any type of adaptor that enable such coupling with the robotic arm 302 as known in the art can be suitable.

When the positioning tool includes an endoscope or a catheter, the endoscope or the catheter can be robotically controlled using the robotic positioning system 300. Each one of the first and second magnetic implants 12, 14 can include a connecting member connectable to a corresponding connector extending from a corresponding endoscope or corresponding catheter to be releasably engageable with the connector. It is to be understood that the drawings and descriptions herein are proffered by way of example only, and that other types of connector and connecting member can also be suitable to enable the connection of the magnetic implant with a positioning tool, which in this case could be referred to as a delivery device, such as an endoscope or a catheter, so that the magnetic implant can be robotically delivered to the site of the desired anastomosis.

Once the magnetic implants are robotically delivered within their respective hollow organ and on their respective side the of the desired anastomosis, the first and second magnetic implants can be brought in close proximity, for instance robotically, to enable magnetic coupling of the first and second magnetic implants through the two adjacent vessel walls of the digestive tract, such that the compression surface of each of the first and second magnetic implants contacts the interior wall of their respective hollow organ at the site of the desired anastomosis. The magnetic coupling of the two magnetic implants compresses a portion of the two adjacent walls therebetween, and the portion that is compressed between the respective compression surfaces of the magnetic implants eventually forms a necrotic area as the blood flood supply to this area progressively declines.

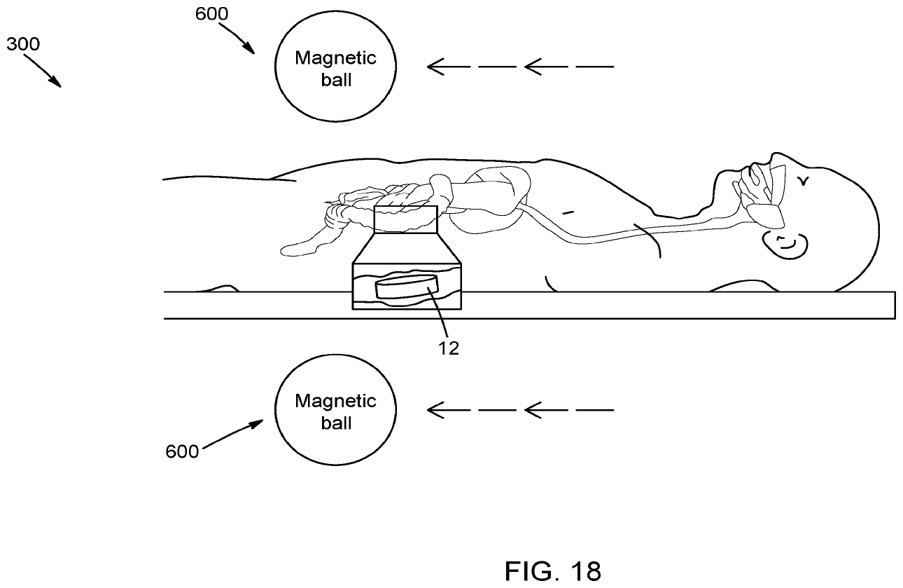

Referring to FIG. 18, in alternative implementations, one of the first and second magnetic implants 12, 14 can be introduced in the digestive tract of the patient, for instance by having the patient swallow the magnetic implant, and the navigation of the magnetic implant to the site of the desired anastomosis can be performed using an external robotic system that is configured to control the movement of the magnetic implant once swallowed by the patient. The external robotic system includes a first external magnetic control assembly comprising a first magnetic field generation device configured to be placed on a first side of the patient, and a second external magnetic control assembly comprising a second magnetic field generation device configured to be placed on a second side of the patient. In FIG. 18, the magnetic field generation devices are shown as magnetic balls 600. In some implementations, the two magnetic generation field means, which can be placed above and below the patient once the patient has laid down on a substantially horizontal platform (such as shown in FIG. 18), or can be placed in front and in back of the patient if the patient is standing up. The external magnetic control assemblies provide power to move the magnetic generation means'position and magnetization direction with respect to the patient, in order to provides optimal magnetic field guidance to the magnetic implant. Such external system is described for instance in granted U.S. Pat. Nos. 10,779,709, 10,076,234, and 11,272,858, which are incorporated by reference herein in their entirety.

The external robotic system is configured to perform three-dimensional translational and two-dimensional rotational control to precisely guide the magnetic implant at the site of the desired anastomosis.

### Method for Forming an Anastomosis in the Digestive Tract

A method for forming an anastomosis between two adjacent walls of a digestive tract of a patient will now be described in further detail. The method can include robotically navigating a first magnetic implant into the digestive tract of a patient to a first location, on one side of a desired anastomose site, within the lumen of a first hollow organ, and navigating, optionally robotically, a second magnetic implant into the digestive tract of the patient to a second location on another side of the desired anastomose site, within the lumen of a second hollow organ.

It is to be noted that when the first magnetic implant is robotically navigated or robotically delivered to the first location, the second magnetic implant (or inversely) can be navigated or delivered to the second location according to the same technique, i.e., robotically, or the second magnetic implant can be navigated or delivered to the second location according to a different method, for instance without robotic assistance.

In some implementations, the robotic navigation of the magnetic implant can be performed via a natural cavity of the patient, i.e., the mouth or the anus, using for example an endoscopic device. Alternatively, in some implementations, at least one of the first and second magnetic implants can be robotically navigated to the site of the desired anastomosis using a robotic laparoscopic procedure.

The robotic positioning system can include various features that enable manipulating the positioning tool, such as a positioning wand, an endoscope or a delivery catheter, according to five or more degrees of freedom to facilitate the positioning of the first and second magnetic implants at the desired first location and at the desired second location.

In some implementations, robotically navigating at least one of the first and second magnetic implants comprises robotically controlling a positioning tool engaged with the robotic arm. In some implementations, robotically navigating the at least one of the first and second magnetic implants comprises releasably engaging the first magnetic implant with the positioning tool. In some implementations, the positioning tool comprises an endoscope or a delivery catheter.

In some implementations, a positioning of at least one of the first and second magnetic implants can be robotically adjusted by magnetically coupling, through one of the two adjacent walls, at least one of the first and second magnetic implants with a distal tip moveably engaged with a distal end of an elongated member of a positioning wand, the positioning wand being operatively engaged with a robotic arm of the robotic positioning system. The robotic arm can then guide the positioning wand so that the distal tip surfs, or glides, onto an outer surface of the one of two adjacent walls to translate the at least one of the first and second magnetic implants, the distal tip being movable relative to a distal end of the elongated member in response to a contact pressure upon contact with the outer surface of the one of two adjacent walls. In some implementations, surfing the distal tip onto the outer surface of the one of two adjacent walls comprises gliding the distal tip along the outer surface of the one of two adjacent walls. In other implementations, surfing the distal tip onto the outer surface of the one of two adjacent walls comprises rolling the distal tip along the outer surface of the one of two adjacent walls, for instance when the distal tip includes a cylinder as shown in FIGS. 10 to 13. As used herein, the term “gliding” is intended to refer to the act of being carried along lightly onto a surface, almost as if the distal tip was floating onto the surface. The terms “skimming”, “wafting” and “hovering” are also considered within the intended scope of the term “surfing” to designate the light contact between the distal tip of the positioning wand and the outer surface of the wall of the organ of the digestive tract, in contrast with “milking”.

Alternatively, in some implementations, the method for positioning at least one of first and second magnetic implants configured for forming an anastomosis at a target site between two adjacent walls of a digestive tract of a patient can include deploying the first magnetic implant into a first hollow organ lumen of the digestive tract, deploying the second magnetic implant into a second hollow organ lumen of the digestive tract, robotically inserting a distal tip and at least a portion of an elongated member of a positioning wand into an abdominal cavity of the patient, the distal tip being movably engaged with a distal end of the elongated member and comprising a guide magnet, magnetically coupling the distal tip of the positioning wand with the second magnetic implant, and robotically gliding, or surfing, the distal tip onto an outer surface of the second hollow organ while maintaining the magnetic coupling with the second magnetic implant to translate the second magnetic implant and bring the second magnetic implant in close proximity with the first magnetic implant to magnetically couple the first magnetic implant with the second magnetic implant.

Once the magnetic implants are delivered within their respective hollow organ and on their respective side the of the desired anastomosis, the first and second magnetic implants can be brought in close proximity to enable magnetic coupling of the first and second magnetic implants through the two adjacent vessel walls of the digestive tract, such that the compression surface of each of the first and second magnetic implants contacts the interior wall of their respective hollow organ at the site of the desired anastomosis. The magnetic coupling of the two magnetic implants compresses a portion of the two adjacent walls therebetween, and the portion that is compressed between the respective compression surfaces of the magnetic implants eventually forms a necrotic area as the blood flood supply to this area progressively declines.

In some implementations, the first and second magnetic implants can be manipulated by using a magnet externally, for instance to facilitate the passing of the coupled magnetic implants via the bowel lumen of the patient once the healing time period is completed. An endoscope can also be used to manipulate the coupled magnetic implants internally, also to facilitate their passing via the bowel lumen of the patient once the healing time period is completed.

Several alternative implementations and examples have been described and illustrated herein. The implementations of the technology described above are intended to be exemplary only. A person of ordinary skill in the art would appreciate the features of the individual implementations, and the possible combinations and variations of the components. A person of ordinary skill in the art would further appreciate that any of the implementations could be provided in any combination with the other implementations disclosed herein. It is understood that the technology may be embodied in other specific forms without departing from the central characteristics thereof. The present implementations and examples, therefore, are to be considered in all respects as illustrative and not restrictive, and the technology is not to be limited to the details given herein. Accordingly, while the specific implementations have been illustrated and described, numerous modifications come to mind.

1. A system for forming an anastomosis between two adjacent walls of a digestive tract, the system comprising:
first and second magnetic implants configured to magnetically couple to each other through the two adjacent walls of the digestive tract to compress a portion of the two adjacent walls therebetween and form a necrotic area that becomes surrounded by a scarred edge following a healing time period;
a positioning tool comprising:
a positioning wand configured for laparoscopic positioning of at least one of the first and second magnetic implants, the positioning wand comprising:
an elongated member sized and configured to have at least a portion thereof to be inserted into an abdominal cavity of a patient; and
a distal tip provided at a distal end of the elongated member and comprising a guide magnet configured to magnetically couple with the at least one of the first and second magnetic implants through one of the two adjacent walls, the distal tip being configured to surf onto an outer surface of the one of the two adjacent walls and be moveable in response to a contact pressure upon contact therewith to translate the at least one of the first and second magnetic implants to a desired site of the anastomosis via the robotic arm;
a robotic positioning system comprising a robotic arm engageable with the positioning wand to impart movement on the distal tip to enable said surfing onto the outer surface of the one of the two adjacent walls.

2. The system of claim 1, wherein the first and second magnetic implants are elongated magnetic implants.

3. The system of claim 1, wherein the positioning tool comprises a plurality of positioning tools, and wherein the plurality of positioning tools comprises an endoscope or a delivery catheter, and the positioning wand.

4. (canceled)

5. (canceled)

6. The system of claim 1, wherein the robotic arm comprises first and second robotic arms each comprising a corresponding positioning tool for delivery of the first and second magnetic implants respectively.

7. The system of claim 1, wherein the robotic arm comprises articulations to facilitate guiding the distal tip of the positioning wand of the magnetic implant along the digestive tract to the desired site of the anastomosis by surfing the distal tip onto the outer surface of the one of the two adjacent walls.

8. The system of claim 1, wherein the distal tip is pivotally engaged with the elongated member via a pin, the distal tip forming a hinge pivoting back and forth about the pin.

9. The system of claim 1, wherein the distal tip is pivotally engaged with the elongated member via a pin, the distal tip having a cylindrical shape with a central cylindrical longitudinal axis coinciding with the pin such that the distal tip is rotatable about the central cylindrical longitudinal axis.

10. The system of claim 1, wherein the distal tip comprises a frame pivotally engaged with the distal end of the elongated member via a pin, the frame forming a hinge pivoting back and forth about the pin.

11. The system of claim 10, wherein the distal tip comprises at least one cylindrical body pivotally engaged with the frame via a cylindrical body pin extending along a central cylindrical body longitudinal axis of a corresponding cylindrical body such that the cylindrical body is rotatable about the central cylindrical longitudinal axis.

12. The system of claim 1, wherein the distal tip is engaged with the elongated member via a spherical swiveling joint.

13. The system of claim 1, wherein the distal tip is engaged with the distal end of the elongated member via a biasable connection.

14. A method for forming an anastomosis between two adjacent walls of a digestive tract of a patient with a robotic positioning system, the method comprising:
robotically navigating a first magnetic implant into a first hollow organ of the digestive tract to a first location on one side of a desired anastomose site;
robotically navigating a second magnetic implant into a second hollow organ of the digestive tract to a second location on another side of the desired anastomose site;
robotically adjusting a positioning of at least one of the first and second magnetic implants, comprising:
magnetically coupling, through one of the two adjacent walls, at least one of the first and second magnetic implants with a distal tip moveably engaged with a distal end of an elongated member of a positioning wand, the positioning wand being operatively engaged with a robotic arm of the robotic positioning system;
surfing the distal tip onto an outer surface of the one of two adjacent walls to translate the at least one of the first and second magnetic implants, the distal tip being movable relative to a distal end of the elongated member in response to a contact pressure upon contact with the outer surface of the one of two adjacent walls; and
magnetically coupling the first and second magnetic implants to each other through the two adjacent vessel walls of the digestive tract to compress a portion of the two adjacent walls therebetween and form a necrotic area during a healing time period.

15. The method of claim 14, wherein robotically navigating at least one of the first and second magnetic implants comprises robotically controlling a positioning tool engaged with the robotic arm.

16. (canceled)

17. The method of claim 15, wherein the positioning tool comprises an endoscope or a delivery catheter.

18. (canceled)

19. The method of claim 14, wherein surfing the distal tip onto the outer surface of the one of two adjacent walls comprises gliding the distal tip along the outer surface of the one of two adjacent walls.

20. The method of claim 14, wherein surfing the distal tip onto the outer surface of the one of two adjacent walls comprises rolling the distal tip along the outer surface of the one of two adjacent walls.

21. A method for positioning at least one of first and second magnetic implants configured for forming an anastomosis at a target site between two adjacent walls of a digestive tract of a patient, the method comprising:
deploying the first magnetic implant into a first hollow organ lumen of the digestive tract;
deploying the second magnetic implant into a second hollow organ lumen of the digestive tract;
robotically inserting a distal tip and at least a portion of an elongated member of a positioning wand into an abdominal cavity of the patient, the distal tip being movably engaged with a distal end of the elongated member and comprising a guide magnet;
magnetically coupling the distal tip of the positioning wand with the second magnetic implant; and
robotically surfing the distal tip onto an outer surface of the second hollow organ while maintaining the magnetic coupling with the second magnetic implant to translate the second magnetic implant and bring the second magnetic implant in close proximity with the first magnetic implant to magnetically couple the first magnetic implant with the second magnetic implant.

22. The method of claim 21, wherein robotically inserting the distal tip and the at least a portion of the elongated member of the positioning wand into the abdominal cavity of the patient is performed laparoscopically via a robotic arm.

23. (canceled)

24. (canceled)

25. The method of claim 21, wherein surfing the distal tip onto the outer surface of the one of two adjacent walls comprises gliding the distal tip along the outer surface of the one of two adjacent walls.

26. The method of claim 21, wherein surfing the distal tip onto the outer surface of the one of two adjacent walls comprises rolling the distal tip along the outer surface of the one of two adjacent walls.

27.-32. (canceled)

33. A robotic positioning system for positioning at least one of first and second magnetic implants configured for forming an anastomosis at a target site between two adjacent walls of a digestive tract of a patient, the robotic positioning system comprising:
a positioning wand comprising:
an elongated member sized and configured to have at least a portion thereof to be inserted laparoscopically into an abdominal cavity of a patient; and
a distal tip provided at a distal end of the elongated member and comprising a guide magnet configured to magnetically couple with the at least one of the first and second magnetic implants through one of the two adjacent walls, the distal tip being configured to surf onto an outer surface of the one of the two adjacent walls and be moveable in response to a contact pressure upon contact therewith to translate the at least one of the first and second magnetic implants to a desired site of the anastomosis via the robotic arm; and
a robotic arm engageable with the positioning wand to impart movement on the distal tip to enable said surfing onto the outer surface of the one of the two adjacent walls.

34. The system of claim 33, wherein the first and second magnetic implants are elongated magnetic implants.

35. The system of claim 34, wherein the elongated magnetic implants have an aspect ratio of about 1:2 to 1:40.

36. The system of claim 33, wherein the robotic positioning system further comprises a plurality of positioning tools, and wherein the plurality of positioning tools comprises an endoscope or a delivery catheter, and the positioning wand.

37. The system of claim 36, wherein the plurality of positioning tools further comprises a secondary laparoscopic grasper tool.

38. (canceled)

39. The system of claim 33, wherein the robotic arm comprises first and second robotic arms each comprising a corresponding positioning tool for delivery the first and second magnetic implants respectively.

40. The system of claim 33, wherein the robotic arm comprises articulations to facilitate guiding the distal tip of the positioning wand of the magnetic implant along the digestive tract to the desired site of the anastomosis by surfing the distal tip onto the outer surface of the one of the two adjacent walls.

41. The system of claim 33, wherein the distal tip is pivotally engaged with the elongated member via a pin, the distal tip forming a hinge pivoting back and forth about the pin.

42. The system of claim 33, wherein the distal tip is pivotally engaged with the elongated member via a pin, the distal tip having a cylindrical shape with a central cylindrical longitudinal axis coinciding with the pin such that the distal tip is rotatable about the central cylindrical longitudinal axis.

43. (canceled)

44. (canceled)

45. (canceled)

46. (canceled)
